(19) United States
(12) Patent Application Publication
(10) Pub. No.: US 2018/0000517 A1
(43) Pub. Date: Jan. 4, 2018

(54) ANTI-ADHESION INTRAUTERINE BALLOON

(71) Applicant: JMD Innovation Inc., Burnaby (CA)

(72) Inventors: Lina HU, Burnaby (CA); Huafeng LI, Burnaby (CA); Ji ZHANG, Burnaby (CA)

(21) Appl. No.: 15/520,830

(22) PCT Filed: Oct. 22, 2015
(86) PCT No.: PCT/CA2015/051074

§ 371 Date: Apr. 20, 2017

Related U.S. Application Data
(60) Provisional application No. 62/067,041, filed on Oct. 22, 2014.

(51) Int. Cl. A61B 17/42; A61M 25/10; A61M 25/00; A61B 17/02; A61B 17/00

(52) U.S. Cl. CPC A61B 17/42; A61M 25/1002; A61M 25/0045; A61M 2025/0056; A61B 2017/00557; A61B 2017/0225; A61B 2017/4216; A61M 2025/1047; A61M 2210/1433

(57) ABSTRACT

An intrauterine balloon catheter for inhibiting formation of intrauterine adhesions in a mammalian uterus comprising balloon and a sheath, wherein the balloon is at least partially attached to the inner surface of the sheath and wherein the balloon and sheath, when the balloon is inflated, conform to the size and shape of the uterus and contact the wall of the uterus. The catheter further comprises a pressure control lock, clipping unit and one or more therapeutic agents which are therapeutically effective in inhibiting intrauterine adhesion formation.

### CROSS-REFERENCE TO RELATED APPLICATIONS

This application claims the benefit of U.S. Provisional Application No. 62/067,041, filed Oct. 22, 2014, the contents of which are incorporated herein by reference in their entirety.

### TECHNICAL FIELD

The subject matter described herein relates to an intrauterine balloon catheter for the purpose of inhibiting or preventing the formation of intrauterine adhesions. The presently disclosed devices can be introduced into a uterus which is diseased or which has undergone trauma such as fibroid removal or termination of a pregnancy. The balloon catheter is inflated to conform to the size and shape of the uterus. In some embodiments, the outer surface of the catheter device is coated with a therapeutic agent effective to prevent adhesions.

### BACKGROUND

The unwanted adherence of tissues to each other following medical intervention, an event termed an adhesion, is a complication that can lead to painful and debilitating medical problems. The presence of adhesions within the uterine cavity can lead to infertility. Surgical resection of these adhesions has a high rate of adhesion re-formation due to the close proximity of the uterine walls. The universal incidence of intrauterine adhesions (IUA) is steadily increasing as any factor leading to destruction of the endometrium may engender adhesions of the myometrium at opposing walls of the uterus as after myomectomy and uterine septum excision. The main etiologic factor is trauma to a recently pregnant uterus and the incidence is high in countries with increased therapeutic and illegal abortions, also in areas with high incidence of genital tuberculosis. Certain patients develop severe form of IUA and others are unaffected while undergoing the same traumatic procedures. This concept may also explain why some patients respond well to treatment whereas others suffer from recurrent adhesions. The diagnosis of IUA is made by hystersalpingography, and mainly by hysteroscopy. Hysteroscopy has become accepted as the optimum route of surgery, the aims being to restore the size and shape of the uterine cavity, normal endometrial function and fertility. Treatment can range from simple cervical dilatation in the case of cervical stenosis but an intact uterine cavity, to extensive adhesiolysis of dense intrauterine adhesions using scissors or electro- or laser energy. The success of treatment regarding term deliveries and rate of abortions depends on the severity of the adhesions. Patients in whom the uterine fundus is completely obscured and those with a greatly narrowed, fibrotic cavity present the greatest therapeutic challenge. Several techniques have described for these difficult cases, but outcome is far worse than in patients with mild, endometrial-type adhesions. Non-hysteroscopic techniques area also beginning to be developed, but whether they will replace the current ‘gold’ standard of hysteroscopy remains to be seen.

There does not appear to be a define uniform approach to prevent occurrence of intrauterine adhesions and the topics of intrauterine contraceptive device (IUCD) insertion, Balloon Catheter, Estrogen/Estrogen and Progestins, Antibiotics/Corticosteroids are constantly debated. The use of an inflated pediatric Foley catheter balloon in the uterine cavity with its stem coming out of the cervical canal, instead of an IUCD to mechanically maintain the uterine cavity separated after adhesiolysis had been reported with equally good results with fewer complications.

Conventional technologies for preventing intrauterine adhesions have limited effectiveness. Accordingly, described herein is an apparatus for preventing intrauterine adhesions.

The foregoing examples of the related art and limitations related therewith are intended to be illustrative and not exclusive. Other limitations of the related art will become apparent to those of skill in the art upon a reading of the specification and a study of the drawings.

### BRIEF SUMMARY

The following aspects and embodiments thereof described and illustrated below are meant to be exemplary and illustrative, not limiting in scope.

The present disclosure is directed to an intrauterine balloon catheter comprising an inflatable balloon and a flexible sheath, wherein the balloon shape, when inflated, takes on the shape of the site to be controlled (for example, the shape of a uterine wall). An insufflation tube feeds a distending medium (such as water or a physiologic fluid, or alternatively a gas) through a supply tube to inflate the balloon. The apparatus comprises a pressure lock control for maintaining a constant pressure inside the balloon as until the bleeding reduces significantly or stops. The inflated balloon presses against the uterine wall to check the flow of bleeding from the wall. One benefit of the balloon is that it is effective when bleeding occurs at multiple sites in the uterine wall.

In one embodiment, the sheath comprises two or more pieces. In another embodiment, the two or more pieces are partially attached or fixed to each other.

In one embodiment, the sheath comprises a material selected from the group consisting of silicon, natural or synthetic rubber, and fabric. In another embodiment, the material is biocompatible.

In one embodiment, the catheter comprises a clipping unit. In another embodiment, the clipping unit controls the expansion and contraction of the sheath two or more pieces.

In one embodiment, the pressure lock control may take on various forms, each of which produces a gradual discharge of fluid from the balloon to maintain a constant pressure inside of the balloon as necessary to control or stop bleeding. The control includes a series of valves according to devices known in the art. In one aspect of the disclosure, the pressure lock control includes a combination fill and pressure regulating valve. In another aspect of the invention the control includes a combination fill and pressure relief valve. In still another aspect of the invention the control includes a high/low pressure warning device.

In one aspect, the intrauterine balloon catheter comprises one or more therapeutic agents which inhibit or prevent the formation of intrauterine adhesions. In another embodiment, the one or more therapeutic agents is present on the outer surface of the intrauterine balloon catheter sheath.

An advantage provided by the present invention is that its insertion, inflation, and removal require no surgical procedures, and very little time. The device is thus uniquely and admirably suitable for use preventing intrauterine adhesions.

In one aspect is a method for preventing or inhibiting the formation of intrauterine adhesions in a mammalian uterus. In one embodiment, the uterus is diseased or has experienced trauma.

Although the devices as disclosed herein may be used in various surgical procedures, its use will be described in detail only in connection with an improved cesarean section surgical procedure.

### BRIEF DESCRIPTION OF DRAWINGS

FIGS. 1A-1B illustrate non-limiting embodiments of a uterine balloon shape according to the present disclosure.

FIGS. 2A-2C illustrate various embodiments of an intrauterine balloon catheter according to the present disclosure.

FIGS. 3A-3B illustrate at least inflation and deflation of an intrauterine balloon catheter according to the present disclosure.

### DETAILED DESCRIPTION

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Disclosed herein is a uterine device for the prevention of intrauterine adhesions due to disease or trauma. The device comprises a contoured structure of a flexible material having the general contours of an interior cavity of a human female uterus wherein the device comprises a therapeutic agent for treating a uterine condition. In some embodiments, the device comprises at least one release region that controls the release of at least one therapeutic agent (which is disposed beneath or within the release region) for treating or preventing intrauterine adhesions.

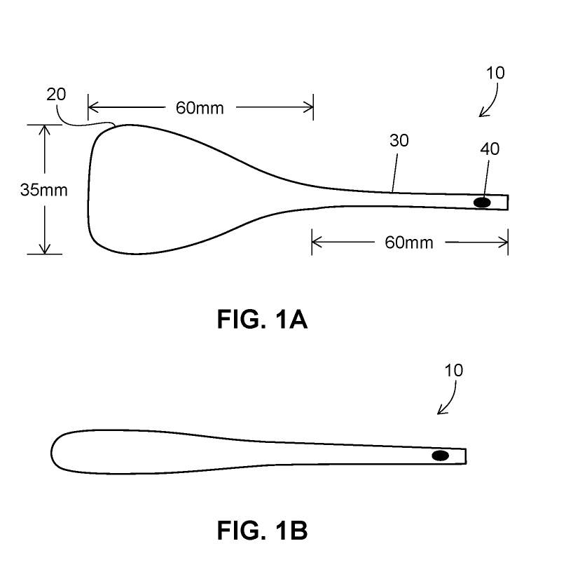

The present invention provides for an improved intrauterine balloon catheter. An intrauterine balloon 10 as used in the presently disclosed device is illustrated in FIG. 1A. Balloon 10 is fabricated from an expandable material, including, but not limited to, polyurethane, silicone, or another medical-grade elastomeric material. Importantly, balloon 10 is fabricated to adopt a shape when inflated which conforms to the shape of the uterus. In one embodiment, balloon 10 in an inflated configuration comprises a main body 20 and a neck portion 30. In some embodiments the length of main body 20 ranges from about 20 to 90 mm or is about 50, 60, or 70 mm. FIG. 1A illustrates a non-limiting embodiment of a main body length of about 60 mm. In other embodiments, the length of neck 30 ranges from about 20 to 90 mm or is about 50, 60, or 70 mm. FIG. 1A illustrates a non-limiting embodiment of a neck length of about 60 mm. The diameter of main body 20 ranges from about 10 to 80 mm or is about 25, 35 or 45 mm. FIG. 1A illustrates a non-limiting embodiment of a main body diameter of about 35 mm.

Located at approximately the proximal end of neck 30 is a pressure lock mechanism 40 which provides control of air or fluid inflow to and outflow from balloon 10. The primary purpose of the pressure lock is to maintain a constant pressure inside balloon 10. Another purpose is to prevent the pressure inside of balloon 10 from exceeding a level which could cause premature ejection of the balloon or injure the uterus. In a normal operating mode, a desired pressure which is less than a maximum allowable pressure is maintained constant by gradually discharging some of the physiologic fluid from single layer balloon if necessary in order to allow the uterus to resume its normal physiologic function. FIG. 1B shows a top view of balloon 10.

The pressure lock mechanism is configured as is readily apparent to the ordinarily skilled artisan. In one embodiment, the pressure lock mechanism comprises a ball wherein pressure may be released by deforming the balloon. As a non-limiting example, a pressure relief valve is located close to the uterus to assure that the pressure in the balloon will not exceed the maximum limit under all conditions, such as pinched tube or a mal functioning pressure regulator valve. Numerous known types of relief valves, such as a ball check valve are available for preventing the pressure from exceeding the maximum limit. A generally cylindrical shaped valve is comprised of a thin resilient wall housing having a pair of flat walls which abut each other to seal the valve when the pressure is below the maximum limit separate and release fluid when the pressure is at the maximum limit. The thickness and diameter of the walls allow the walls to separate and open the valve along the horizontal axis and discharge fluid when the pressure is at the maximum limit.

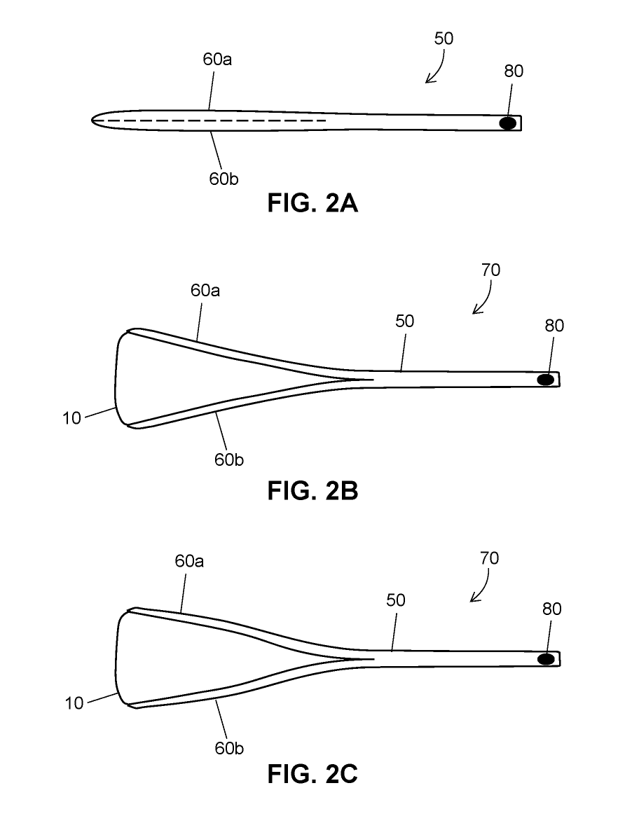

Balloon 10 is attached to the inner surface of a delivery sheath 50 (see FIG. 2). Delivery sheath 50 is comprised of a soft, pliable and/or flexible material including but not limited to silicon, plastic, natural or synthetic rubber, or fabric. In a preferred embodiment, the material is biocompatible. Sheath 50 in some embodiments is comprised of 2 or more pieces (e.g., 60a and 60b in FIGS. 2A-2C) which are partially fixed to one another or which are independent but which are fastened together with a fastener. Accordingly, when the 2 or more pieces are in a compact condition as shown in FIG. 2A, the sheath possess a rigidity which is sufficient to allow insertion into the vagina and delivery of the distal end of the device into the uterus. After delivery into the uterus, an insufflation tube feeds a distending medium (such as water or a physiologic fluid, or alternatively a gas) through a supply tube to inflate balloon 10. As balloon 10 inflates, sheath 50 2 or more pieces 60a and 60b are able to flair laterally, eventually coming in contact with the uterine wall. FIGS. 2A-2C also illustrate a pressure lock 80.

FIG. 2B illustrates the conformation of sheath 50 2 or more pieces 60a and 60b of an intrauterine balloon catheter 70 after partial inflation of balloon 10, prior to contact of sheath 50 2 or more pieces 60a and 60b with the uterine wall. Upon full inflation, as shown in FIG. 2C, sheath 50 2 or more pieces 60a and 60b have a slightly convex shape wherein the outer surfaces of sheath 50 2 or more pieces 60a and 60b conform to and are in contact with the uterine wall.

In one embodiment, sheath 50 is comprised of a shape memory metal. In another embodiment, sheath 50 is comprised of a metal, such as a shape memory metal, which is covered by or coated with a biocompatible plastic material.

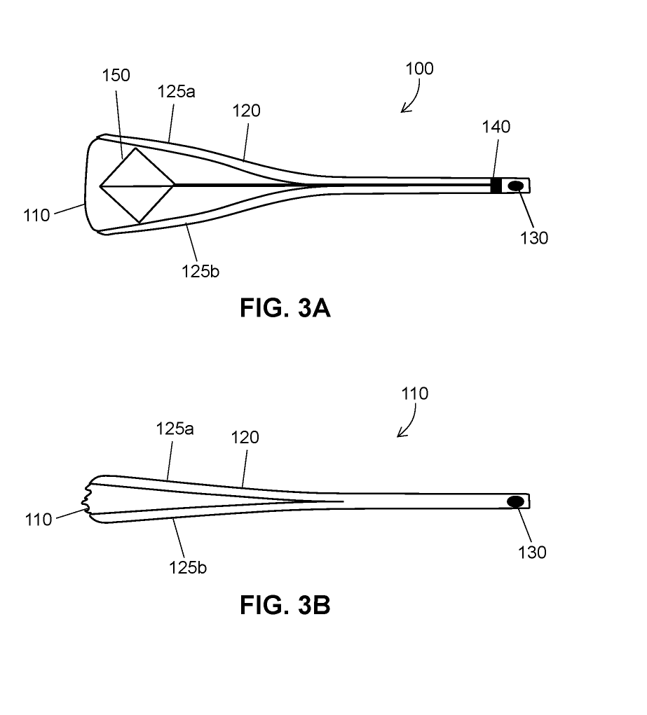

In one embodiment, as illustrated in FIGS. 3A and 3B, intrauterine balloon catheter 110 comprises a balloon 110, a sheath 120 comprising 2 or more pieces 125a and 125b, a pressure lock 130 and a split control 140. Split control 140 ensures that sheath 120 2 or more pieces 125a and 125b are maintained in a compact configuration during delivery into the uterus. After intrauterine balloon catheter 110 is fully delivered into the uterus, split control 140 is manipulated to allow sheath 120 2 or more pieces 125a and 125b to freely expand away from the central axis. After treatment is completed, fluid or air within inflated balloon 110 is removed and sheath 120 2 or more pieces 125a and 125b are brought back to a compact configuration.

In one embodiment, illustrated in FIG. 3A, intrauterine balloon catheter 110 comprises a split control unit 140 comprising an expansion frame 150. Split control unit 140 with expansion frame 150 function to control at least the rate of expansion of the balloon and the shape of balloon 110 as it inflates. In one embodiment, split control until 140 may further control the shape of expanded balloon catheter 110.

In one aspect of the present disclosure, the sheath of the intrauterine balloon catheter is coated with or otherwise comprises one or more therapeutic agent which can inhibit or prevent formation of intrauterine adhesions. This embodiment is useful to ensure that intrauterine adhesions do not form after removing of the catheter device. In one embodiment, the sheath comprises a release region which contains the one or more therapeutic agents. At least one release region also contains at least one polymer that may be selected from any biocompatible polymers that are suitable for use in implantable or insertable uterine medical devices and are suitable for therapeutic agent release. The polymers may be substantially non-biodegradable or biodegradable and a device may comprise a plurality of biocompatible polymers.

An advantage of the uterine devices of the present invention is that since the therapeutic agent is released from the devices in situ, treatment is more direct, and less medication may be required as compared with typical systemic treatments such as oral contraceptives that may cause undesirable side effects. In particular, given that the devices are shaped to generally conform to the contours of the uterine cavity (e.g., the device comes into contact with the walls of the uterine cavity), an appreciable percentage of a surface of the device comes into contact with the uterine wall, facilitating direct and targeted release of the therapeutic agent into the uterine site.

Other optional therapeutic agents may include, for example, antimicrobial agents that have been added in an amount effective to inhibit the growth of microbes on or around the device as well as any antimicrobial agents added for therapeutic purposes. Antimicrobial agents include, for example, triclosan, chlorhexidine and combinations thereof. In some embodiments, a lubricious hydrophilic coating may be applied to the surface of the urological devices and uterine devices of the present disclosure.

Another advantage of the uterine devices of the present disclosure is that therapy using such devices is less invasive than surgical treatment options such as hysterectomy and myolysis.

Another advantage of the intrauterine device of the present disclosure is that since the device may be easily removed and replaced, the type or amount of therapeutic agent may be adjusted accordingly during the duration of the treatment, e.g., a succession of different hormonal agents and/or different agent concentrations may be employed to coincide with the patient's menstrual cycle.

The release regions of the present disclosure form at least a portion of the implantable or insertable medical devices of the present disclosure, and in some instances form the entire medical devices, in which case the materials forming the release region are selected to provide mechanical properties consistent with the intended function and operation of the implantable or insertable medical devices. A device may have a plurality of release regions. The base polymer can also be biodegradable to eliminate the need for removal.

Hence, in certain embodiments, release regions for use in accordance with the present invention are in the form of a release layers, which cover all or a part of a medical device substrate. As used herein a “layer” of a given material is a region of that material whose thickness is small compared to both its length and width. As used herein a layer need not be planar or conformal (for example, taking on the contours of an underlying substrate). Layers can be discontinuous (e.g., patterned). Terms such as “film,” “layer” and “coating” may be used interchangeably herein.

The mixture of polymer and additives can be shaped into at least a portion of a medical device in accordance with the present invention by means of any process conventionally used to shape polymeric materials such as thermoplastic and elastomeric materials. Among such shaping processes are included, but not limited to, extrusion including coextrusion, molding, calendaring, casting and coating.

Preferred substantially non-biodegradable biocompatible polymers include thermoplastic and elastomeric polymeric materials. Polymers for use in the devices of the present invention can be selected, for example, from polyolefins such as metallocene catalyzed polyethylenes, polypropylenes, and polybutylenes and copolymers thereof; vinyl aromatic polymers such as polystyrene; vinyl aromatic copolymers such as styrene-isobutylene copolymers and butadiene-styrene copolymers; ethylenic copolymers such as ethylene vinyl acetate (EVA), ethylene-methacrylic acid and ethylene-acrylic acid copolymers where some of the acid groups have been neutralized with either zinc or sodium ions (commonly known as ionomers); polyacetals; chloropolymers such as polyvinylchloride (PVC); fluoropolymers such as polytetrafluoroethylene (PTFE); polyesters such as polyethyleneterephthalate (PET); polyester-ethers; polyamides such as nylon 6 and nylon 6,6; polyamide ethers; polyethers; elastomers such as elastomeric polyurethanes and polyurethane copolymers; silicones; polycarbonates; and mixtures and block or random copolymers of any of the foregoing are non-limiting examples of non-biodegradable biocompatible polymers useful for manufacturing the medical devices of the present invention.

The intrauterine devices of the present disclosure may also contain a radio-opacifying agent within its structure. For example, the radio-opacifying agent may be present in or on any of the carrier or barrier regions found in the devices. The radio-opacifying agent facilitates viewing of the medical device during insertion of the device and at any point while the device is implanted. A radio-opacifying agent typically functions by scattering x-rays. The areas of the medical device that scatter the x-rays are detectable on a radiograph. Among radio-opacifying agents useful in the medical device of the present invention are included, but not limited to, bismuth subcarbonate, bismuth oxychloride, bismuth trioxide, barium sulfate, tungsten and mixtures thereof Where present, the radio-opacifying agent is preferably present in an amount of from about 0.5% to about 90%, more preferably from about 10% to about 90% by weight, of the polymer. A particularly preferred amount of radio-opacifying agent is from about 10 to about 40% by weight of the polymer.

In one embodiment, the intrauterine balloon catheter can be imaged using ultrasound. Ultrasound analysis is useful in confirming proper shape of the balloon in its inflated configuration.

In one aspect of the present disclosure, a method for preventing intrauterine adhesions is provided. For example, a woman having undergone a procedure to terminate a pregnancy or to remove fibroids is at risk for the formation of intrauterine adhesions. Accordingly, the intrauterine balloon catheter as described herein is used to insert the sheath encasing the balloon and inflating the balloon until the outer surface of the sheath is in contact with the uterine wall. Preferably, the inflated balloon/sheath shape conforms to the shape of the uterus. The balloon/sheath remains in the uterus for 1-30 days to ensure no intrauterine adhesions form after removal of the device.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

1. (canceled)

2. (canceled)

3. (canceled)

4. (canceled)

5. (canceled)

6. (canceled)

7. (canceled)

8. (canceled)

9. (canceled)

10. (canceled)

11. An apparatus for preventing intrauterine adhesions, the apparatus comprising:
an intrauterine balloon catheter, comprising:
a flexible sheath having an inner lumen comprising at least one longitudinal perforation or slotted section for permitting the sheath to split into opposing longitudinal pieces from a compact configuration; and
an inflatable balloon disposed within the sheath inner lumen and coupled to a surface of the sheath inner lumen, the balloon having an inflated configuration in which the balloon expands the opposing longitudinal pieces from the compact configuration to conform to a shape of an obstetrical site;
an insufflation tube coupled to the balloon for providing a distending medium to inflate the balloon to the inflated configuration; and
a pressure lock mechanism coupled to a proximal end portion of the catheter and in fluid communication with the balloon for maintaining a constant pressure of the balloon until bleeding reduces significantly or stops.

12. The apparatus of claim 11, wherein the sheath comprises two or more pieces.

13. The apparatus of claim 12, wherein the two or more pieces are partially attached or fixed to each other.

14. The apparatus of claim 11, wherein the pressure lock mechanism permits gradual discharge of fluid from the balloon to maintain a constant pressure inside of the balloon as necessary to control or stop bleeding.

15. The apparatus of claim 14, wherein the intrauterine balloon catheter comprises one or more therapeutic agents for inhibiting or preventing formation of intrauterine adhesions.

16. The apparatus of claim 15, wherein the one or more therapeutic agents is present on an outer surface of the sheath.

17. The apparatus of claim 11, wherein the apparatus comprises a lubricious hydrophilic coating applied to a surface thereof

18. The apparatus of claim 11, wherein the apparatus comprises at least one release region that controls release of at least one therapeutic agent for treating or preventing intrauterine adhesions.

19. The apparatus of claim 11, wherein the opposing longitudinal pieces comprise two or more separate pieces.

20. The apparatus of claim 11, wherein the opposing longitudinal pieces are partially fixed to one another.

21. The apparatus of claim 11, wherein the opposing longitudinal pieces are separate pieces that are fastened together with a fastener.

22. The apparatus of claim 11, wherein the sheath comprises a shape memory metal.

23. The apparatus of claim 11, further comprising a split control mechanism coupled to the opposing longitudinal pieces and configured to maintain the opposing longitudinal pieces in the compact configuration during delivery.

24. The apparatus of claim 23, wherein the split control mechanism comprises an expansion frame coupled to the balloon catheter, the expansion frame being configured to control a rate of expansion of the balloon.

25. The apparatus of claim 24, wherein the split control mechanism further controls a shape of the balloon in the inflated configuration.

26. A method for minimizing or preventing intrauterine adhesions, the method comprising:
inserting an intrauterine balloon catheter into an obstetrical site of a patient in a compact configuration, the intrauterine balloon catheter comprising a flexible sheath and an inflatable balloon disposed within and coupled to an inner lumen of the sheath, the sheath comprising at least one longitudinal perforation or slotted section for permitting the sheath to split into opposing longitudinal pieces from a compact configuration;
expanding the balloon to an inflated configuration in which the balloon expands the opposing longitudinal pieces from the compact configuration to conform to the shape of the obstetrical site; and
releasing the balloon catheter to reside within the obstetrical site.

27. The method of claim 26, wherein the balloon catheter remains in the obstetrical site for 1-30 days.

28. The method of claim 26, wherein the sheath comprises a shape memory metal.

29. The method of claim 26, wherein the expanding comprises inflating the balloon with a distending medium.

30. The method of claim 29, further comprising maintaining pressure inside of the balloon below a maximum allowable pressure to avoid premature ejection of the balloon catheter or injury to the patient.

31. The method of claim 30, wherein the maintaining pressure comprises gradually discharging some physiologic fluid from the balloon to permit the obstetrical site to resume its normal physiologic function.
